(19) United States
(12) Patent Application Publication
(10) Pub. No.: US 2022/0142647 A1
(43) Pub. Date: May 12, 2022

(54) CUFF CLOSURE CLIPS

(71) Applicant: Covidien LP, Mansfield, MA (US)

(72) Inventors: Jacob C. Baril, Norwalk, CT (US); Saumya Banerjee, Collinsville, CT (US); Kevin M. Desjardin, Prospect, CT (US)

(21) Appl. No.: 17/521,172

(22) Filed: Nov. 8, 2021

Related U.S. Application Data
(60) Provisional application No. 63/111,668, filed on Nov. 10, 2020.

(51) Int. Cl. A61B 17/122; A61B 17/128

(52) U.S. Cl. CPC A61B 17/122; A61B 2017/00004; A61B 17/1285

(57) ABSTRACT

An end effector includes first and second jaws selectively movable about a pivot between a spaced position and an approximated position for grasping tissue. The first jaw includes a first link pivotably engaged at a first end to a first side of a proximal end of the first jaw and pivotably engaged at a second end to a proximal flange of the second jaw. A second link is pivotably engaged at a first end to a second side of the proximal end of the first jaw and spaced a distance from the first link, the second link defining a slot configured to slidingly receive a pin disposed in the second jaw. Movement of the jaws causes the first link to pivot toward the second jaw and causes the second link to slide proximally atop the pin to approximate the jaws in a parallel manner through the entire range of motion.

### CROSS-REFERENCE TO RELATED APPLICATIONS

This application claims the benefit of U.S. Provisional Patent Application No. 63/111,668, filed Nov. 10, 2020, the entire disclosure of which is incorporated by reference herein.

### BACKGROUND

### 1. Technical Field

The present disclosure relates to surgical instruments and, more specifically, to instruments for closing the open end of a hollow organ.

### 2. Discussion of Related Art

During hysterectomy procedures, such as total laparoscopic hysterectomy (TLH) procedures, the uterus and cervix are removed, creating an approximately circular structure at end of the vaginal canal called the vaginal cuff. The closure of the remaining vaginal cuff can be one of the most challenging aspects of the procedure. It is also considered to be one of the barriers to adoption of laparoscopic surgery to those trained in open procedures.

After the uterus is removed it is necessary to close the vaginal cuff while avoiding dehiscence, which is when a wound ruptures along a surgical suture. Currently, vaginal cuff closure is done manually by suturing the cuff with a needle and suture or with a stitching surgical instrument. Both methods require a good deal of surgical skill and time.

### SUMMARY

Provided in accordance with the present disclosure is an end effector including a pair of first and second jaw members each having proximal and distal ends and selectively movable about a pivot between a spaced position for orienting tissue between the jaw members and an approximated position for grasping tissue. The first jaw member includes: a first link pivotably engaged at a first end to a first side of the proximal end of the first jaw member and pivotably engaged at a second end to a proximal flange of the second jaw member; and a second link pivotably engaged at a first end to a second side of the proximal end of the first jaw member and spaced a distance distal to the first link. The second link includes a slot defined therealong configured to slidingly receive a pin disposed in the second jaw member.

Movement of the first jaw member relative to the second jaw member causes the first link to pivot toward the second jaw member and causes the second link to slide proximally atop the pin to approximate the jaw members in a parallel manner relative to one another through the entire range of motion.

In aspects according to the present disclosure, the second jaw member includes a slot defined therein configured to receive the second link during proximal translation thereof. In other aspects according to the present disclosure, the outer peripheral dimension of the jaw members when fully approximated is no greater than the outer peripheral dimension of an elongated shaft configured to support the end effector.

In aspects according to the present disclosure, the distance between the first ends of the first and second links is based on the length of the jaw members to insure parallel closure of the first and second jaw members during approximation.

In aspects according to the present disclosure, the end effector is operably coupled to a distal end of an elongated shaft extending from a housing, the housing including a ratchet mechanism disposed therein configured to control the approximation of the jaw members between the spaced position and the approximated position. In other aspects according to the present disclosure, the ratcheting mechanism is configured to regulate the clamping force between the jaw members.

Provided in accordance with the present disclosure is tissue clip including a pair of first and second jaw members each having proximal and distal ends and selectively movable about a hinge between a spaced position for orienting tissue between opposing surfaces of the jaw members and an approximated position for grasping tissue. The jaw members include complementary mechanical interfaces configured to selectively lock the jaw members in the approximated position. One or more retention features is disposed on the opposing surface of one or both of the jaw members and is configured to facilitate tissue retention when the jaw members are disposed in the approximated position. An anchoring feature is defined through the first jaw member and is configured to facilitate anchoring the clip to the tissue engaged between the jaw members when disposed in the approximated position.

In aspects according to the present disclosure, the tissue clip is made from a biodegradable or bioabsorbable material. In other aspects according to the present disclosure, the anchoring feature includes a plurality of slots defined through the first jaw member.

In aspects according to the present disclosure, the retention feature(s) is selected from the group consisting of ridges, protuberances, recesses, high friction surfaces, texturing, and/or ribs.

In aspects according to the present disclosure, the complementary mechanical interfaces include a flange and slot arrangement configured for snap-fit mechanical engagement. In other aspects according to the present disclosure, the complementary mechanical interfaces are selected from the group consisting of: snap-fit arrangements, tongue and groove arrangements, hook and loop arrangements and complementary eyelets.

In aspects according to the present disclosure, one or both of the jaw members includes a coating configured to promote healing and/or reduce infection. In other aspects according to the present disclosure, the tissue clip is made from a biodegradable or bioabsorbable material infused with chemical components that promotes healing and/or reduces infection.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description given below, serve to explain the principles of the disclosure, wherein:

FIG. 1 is a perspective view of one embodiment of a tissue closure system in accordance with one aspect of the present disclosure including an illustrative tissue approximation device and an illustrative tissue closure device;

FIG. 2A is a schematic side view of a distal portion of the tissue approximation device of the tissue closure system of FIG. 1 show in an open position;

FIG. 2B is a schematic side view of the distal portion of the tissue approximation device of the tissue closure system of FIG. 1 show in an approximated position;

FIG. 2C is a schematic perspective view of the distal portion of a tissue approximation device of the tissue closure system of FIG. 1 shown engaging a distal end of a vaginal cuff;

FIG. 3A is a perspective view of an embodiment of a closure clip configured to close an open end of the vaginal cuff; and

FIG. 3B is a schematic perspective view of the distal portion the closure clip of FIG. 3A shown engaging the distal end of the vaginal cuff.

### DETAILED DESCRIPTION

Embodiments of the present disclosure are described in detail with reference to the drawings, in which like reference numerals designate identical or corresponding elements in each of the several views. As used herein, the term “distal” refers to that portion of structure farther from the user, while the term “proximal” refers to that portion of structure, closer to the user. As used herein, the term “clinician” refers to a doctor, nurse, or other care provider and may include support personnel. In the following description, well-known functions or constructions are not described in detail to avoid obscuring the present disclosure in unnecessary detail.

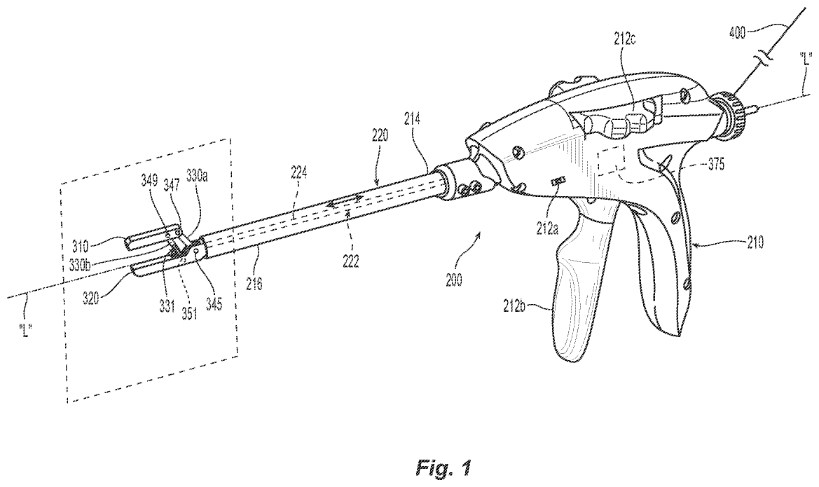

Turning now to FIG. 1, one embodiment of a vaginal tissue closure device 200 includes a handle assembly 210 with one or more actuators 212a, 212b, 212c that may include any suitable slide, knob, button, trigger or the like. The tissue closure device 200 further includes an elongated tubular body portion or shaft 220 that extends distally from the handle assembly 210 and defines a longitudinal axis “L-L” extending through proximal 214 and distal end 216 of the elongated tubular body portion 220. The elongated tubular body portion 220 includes a drive assembly 222 extending therethrough and supports an end effector assembly 300 at the distal end 216 thereof. The drive assembly 222 includes a drive arm 224 coupled to the handle assembly 220 at a proximal end thereof and a cam pin 351 coupled to the end effector assembly 300 at a distal end thereof. The drive arm 224 and cam pin 351 of the drive assembly 222 are axially movable along the longitudinal axis “L-L,” in response to actuation of one or more of the actuators 212a-212c of the handle assembly 210 to remotely operate the end effector assembly 300. The drive arm 224 may be in the form of a rod, tube, shaft, chain, cable, wire, and/or combinations thereof.

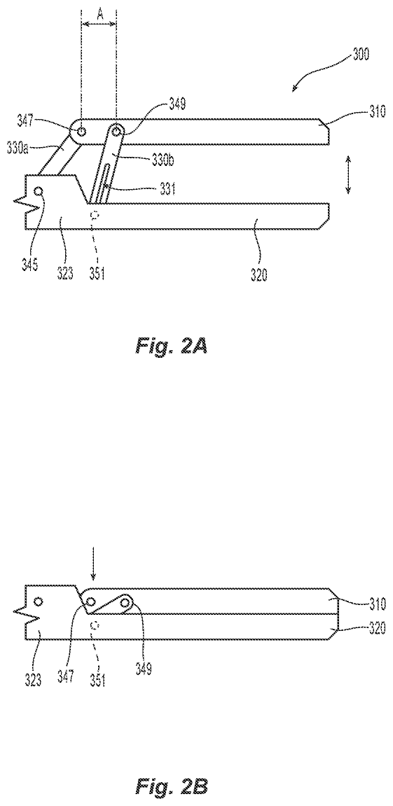

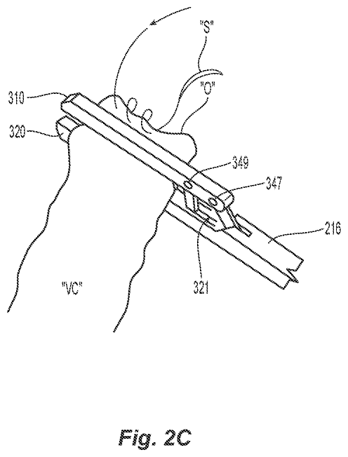

As shown in FIGS. 2A-2C, the end effector assembly 300 of the tissue closure device 200 includes a first jaw member 310 and a second jaw member 320 that are pivotally coupled together about a pivot pin 345 and that are movable relative to one another from an open position (FIG. 2A) and an approximated position (FIG. 2B). One or both of the jaw members 310, 320 may include inner gripping elements or surfaces (not shown) configured to facilitate securing tissue between jaw members 310, 320. Inner gripping surfaces may include one or more teeth, ridges, spikes, texturing, etc. to facilitate tissue gripping. The outer support surface of either or both jaw members 310, 320 may include a suture mounting structure (not shown) configured to selectively support one or more sutures “S”.

As mentioned above, FIG. 2A shows the jaw members 310, 320 of the end effector assembly 300 in an open position for orienting vaginal tissue, e.g., vaginal cuff “VC”. More particularly, jaw member 310 is selectively pivotable about pivot pin 345 via drive arm 224. Jaw member 310 includes a pair of cooperating linkages 330a, 330b that are configured to facilitate movement of jaw member 310 relative to jaw member 320 in a parallel or substantially parallel manner for grasping an opening “O” in vaginal cuff “VC”. Link 330a connects at one end to one side of jaw member 310 via pin 347 and at an opposite end to a proximal flange 323 of jaw member 320 via pivot 345. Link 330b connects at one end to the opposite side of jaw member 310 via pin 349 and at an opposite end to a pin 351 disposed within jaw member 320 (shown in phantom). Link 330b includes a slot 331 defined therein configured to slidingly receive pin 351.

Upon approximation, jaw member 310 is moved relative to jaw member 320 via proximal translation of link 330b via drive arm 224. Upon translation of pin 351, link 330b slides atop pin 351 to control movement thereof, e.g., via pin 351, to insure parallel movement between jaw members 310, 320 during approximation. A slot 321 is defined in jaw member 321 to facilitate translation of link 330b (See FIG. 2C). The distance “A” between the links 330a, 330b where each respective pin 347, 349 connects to jaw member 310 may be adjusted to insure parallel closure of the jaw members 310, 320, e.g., particularly with jaw members 310, 320 of longer or shorter lengths. In other words, the distance “A” may depend on the length of the jaw members 310, 320 for a particular purpose.

By approximating the jaw members 310, 320 in a parallel manner, the opening “O” of the vaginal cuff “VC” may be clamped at the proximal and distal ends of the jaw members 310, 320 simultaneously (or substantially simultaneously) to insure the integrity of the pneumoperitoneum while closing the opening “O” with sutures “S”. Approximation of the jaw members 310, 320 may be regulated by the actuator 212b which may include a ratcheting mechanism 375 to allow the user to set a particular clamping force on the vaginal cuff “VC”. The ratcheting mechanism 375 may also facilitate positioning of the opening “O” during approximation since the jaw members 310, 320, when ratcheted, remain fixed until either released or further approximated allowing the user more freedom during positioning of the tissue between the jaw members 310, 320.

Since the jaw members 310, 320 open and close in a parallel manner, vaginal tissue of varying sizes may be accommodated between the jaw members 310, 320 while still maintaining the pneumoperitoneum across the entire opening of the vaginal cuff “VC”. This facilitates suturing across the entire opening “O” of the vaginal cuff “VC” without having to reposition the instrument which can compromise the integrity of the pneumoperitoneum while suturing.

In aspects according to the present disclosure, the outer peripheral dimension of the jaw members 310, 320 when fully approximated is no greater than the outer peripheral dimension of elongated shaft 220. Moreover, the first and second links 330a, 330b when approximated are configured to fall within the outer peripheral dimensional envelop of the jaw members 310, 320 facilitating insertion through a port or cannula. Orienting the first end of the first link 330a on one side of the first jaw member 310 and orienting the first end of the second link 330b on the opposite side of the first jaw member 310 allows the links 330a, 330b to fall within the dimensional envelop of the jaw members 310, 320 when approximated, promotes lateral stability of the end effector 300 and promotes parallel closure of the jaw members 310, 320.

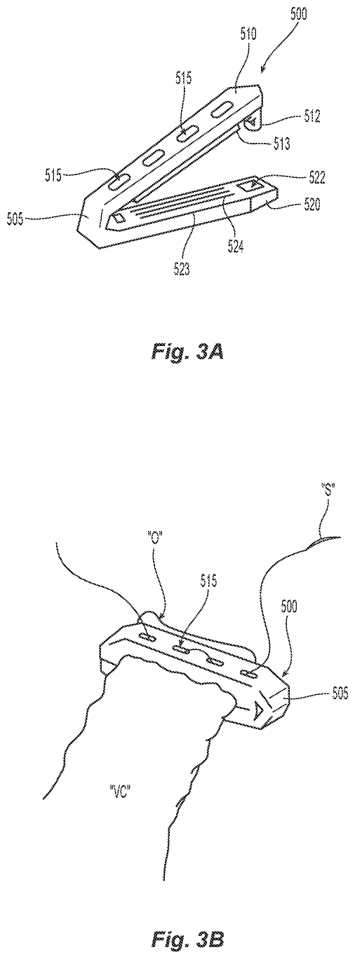

FIGS. 3A and 3B show another embodiment of a vaginal closure device according to the present disclosure represented as closure clip 500. Clip 500 includes jaw members 510, 520 which are pivotably coupled to one another about a living hinge 505. Jaw members 510, 520 are selectively moveable relative to one another from a first, open position for accepting vaginal tissue, e.g., an open end “O” of a vaginal cuff “VC” and a second, approximated position for grasping the opening “O” of the vaginal cuff “VC”. Other types of hinges 505 are envisioned.

Jaw members 510, 520 include respective opposing gripping surfaces 513, 523 that are configured to cooperate to grasp vaginal tissue when the clip 500 is moved to the second position. One or more retention features, e.g., retention features 524, may be included to facilitate grasping and securing vaginal tissue and may include ridges, protuberances, recesses, high friction surfaces, texturing, ribs, etc.

Clip 500 may include one or more locking features to facilitate engagement and retention of the clip 500 atop the vaginal cuff “VC”. For example, jaw member 520 may include one or more slots 522 defined therein configured to operatively engage a corresponding number of opposing flanges 512 extending from jaw member 510. Upon engagement of tissue, e.g., an open end “O” of the vaginal cuff “VC” between opposing grasping surfaces 513, 523, one or both of the jaw members 510, 520 may be moved to approximate the vaginal cuff “VC” such that the locking feature, e.g., slot 522 and flange 512, are selectively engaged to retain the clip 500 in an approximated position (FIG. 3B). The locking feature may include a snap-fit interface, a tongue and groove interface or any other mechanically interfacing components configured to selectively lock the jaw members 510, 520 relative to one another.

In use, once the uterus and ovaries are removed, the surgeon must close the opening “O” in the vaginal cuff “VC” while maintaining the integrity of the pneumoperitoneum. The clip 500 is positioned atop the vaginal cuff “VC” proximate opening “O”, approximated and then selectively locked to close the opening “O”. The unique geometry of the clip 500, e.g., clip length and clip width, insures the entire opening “O” is sufficiently closed to allow a surgeon to suture the opening “O”. In this instance, the surgeon sutures the opening “O” at the distal side of the clip 500 and then removes the clip 500 by unlocking the locking feature, e.g., slot 522 and flange 512 arrangement.

Clip 500 may also be biodegradable or bioabsorbable and configured to remain in situ. For example, clip 500 may include a series of anchoring slots 515 defined through one or both jaw members 510, 520 which allow the surgeon to anchor the clip 500 to the vaginal cuff “VC”, and insure the integrity of the closure. The surgeon simply sutures the clip 500 to the grasped tissue through the anchoring slots 515 when the clip 500 is locked atop the tissue. The clip 500 remains in place and is absorbed by the body over a specific period of time. In this instance, the clip 500 may include beneficial coatings or biodegradable components that promote healing and/or reduce infection. Other types of anchoring features are also envisioned, loops, hooks, eyelets, etc.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed:

1. An end effector, comprising:
a pair of first and second jaw members each having proximal and distal ends and selectively movable about a pivot between a spaced position for orienting tissue between the jaw members and an approximated position for grasping tissue, the first jaw member including:
a first link pivotably engaged at a first end to a first side of the proximal end of the first jaw member and pivotably engaged at a second end to a proximal flange of the second jaw member; and
a second link pivotably engaged at a first end to a second side of the proximal end of the first jaw member and spaced a distance distal to the first link, the second link including a slot defined therealong configured to slidingly receive a pin disposed in the second jaw member,
wherein movement of the first jaw member relative to the second jaw member causes the first link to pivot toward the second jaw member and causes the second link to slide proximally atop the pin to approximate the jaw members in a parallel manner relative to one another through the entire range of motion.

2. The end effector according to claim 1, wherein the second jaw member includes a slot defined therein configured to receive the second link during proximal translation thereof

3. The end effector according to claim 1, wherein the outer peripheral dimension of the jaw members when fully approximated is no greater than the outer peripheral dimension of an elongated shaft configured to support the end effector.

4. The end effector according to claim 1, wherein the distance between the first ends of the first and second links is based on the length of the jaw members to insure parallel closure of the first and second jaw members during approximation.

5. The end effector according to claim 1, wherein the end effector is operably coupled to a distal end of an elongated shaft extending from a housing, the housing including a ratchet mechanism disposed therein configured to control the approximation of the jaw members between the spaced position and the approximated position.

6. The end effector according to claim 5, wherein the ratcheting mechanism is configured to regulate the clamping force between the jaw members.

7. A tissue clip, comprising:
a pair of first and second jaw members each having proximal and distal ends and selectively movable about a hinge between a spaced position for orienting tissue between opposing surfaces of the jaw members and an approximated position for grasping tissue, the jaw members including complementary mechanical interfaces configured to selectively lock the jaw members in the approximated position;
at least one retention feature disposed on the opposing surface of at least one of the jaw members configured to facilitate tissue retention when the jaw members are disposed in the approximated position; and
an anchoring feature defined through the first jaw member and configured to facilitate anchoring the clip to the tissue engaged between the jaw members when disposed in the approximated position.

8. The tissue clip according to claim 7, wherein the tissue clip is made from a biodegradable or bioabsorbable material.

9. The tissue clip according to claim 7, wherein the anchoring feature includes a plurality of slots defined through the first jaw member.

10. The tissue clip according to claim 7, wherein the at least one retention feature is selected from the group consisting of ridges, protuberances, recesses, high friction surfaces, texturing, and ribs.

11. The tissue clip according to claim 7, wherein the complementary mechanical interfaces include a flange and slot arrangement configured for snap-fit mechanical engagement.

12. The tissue clip according to claim 7, wherein the complementary mechanical interfaces are selected from the group consisting of: snap-fit arrangements, tongue and groove arrangements, hook and loop arrangements and complementary eyelets.

13. The tissue clip according to claim 7, wherein at least one of the jaw members includes a coating configured to at least one of promote healing or reduce infection.

14. The tissue clip according to claim 7, wherein the tissue clip is made from a biodegradable or bioabsorbable material infused with chemical components that at least one of promote healing or reduce infection.
